(19) United States
(12) Patent Application Publication
(10) Pub. No.: US 2022/0257521 A1
(43) Pub. Date: Aug. 18, 2022

(54) METHOD FOR MICRO-ENCAPSULATION OF NATURAL INGREDIENTS BY MEANS OF CONTACTING WITH SUPERCRITICAL GAS

(71) Applicant: FOLIUM BIOSCIENCES EUROPE B.V., WEESP (NL)

(72) Inventors: Daniela Oana TRAMBITAS, WEESP (NL); Farnaz SOTOODEH, WEESP (NL); Geert Feye WOERLEE, WEESP (NL)

(73) Assignee: FOLIUM BIOSCIENCES EUROPE B.V., WEESP (NL)

(21) Appl. No.: 17/627,439

(22) PCT Filed: Jun. 15, 2020
(86) PCT No.: PCT/EP2020/066531

§ 371 Date: Jan. 14, 2022

(30) Foreign Application Priority Data
Jun. 14, 2019 (EP) 19180414.5

(51) Int. Cl. A61K 9/48

(52) U.S. Cl. CPC A61K 9/4833; A61K 9/4875; A61K 9/4866; A61K 9/4858

(57) ABSTRACT

The invention provides a method for microencapsulating a natural ingredient in an encapsulation material, comprising:

mixing said natural ingredient for microencapsulation in a first liquid for providing a first mixture;

mixing said encapsulation material in a second liquid for providing a second mixture, said first liquid and second being substantially immiscible, in particular displaying phase separation after being mixed;

preparing a microemulsion from said first mixture and said second mixture using mechanical shear forces;

spraying said microemulsion through a nozzle into a high-pressure vessel while drying it under continuously circulating supercritical CO2 to a powder having a particle size of between 0.1 and 100 microns, in particular between 0.1 and 10 microns.

### FIELD OF THE INVENTION

The invention relates to a method for microencapsulating a natural ingredient in an encapsulation material, the microencapsulated material, and use of such microencapsulated material.

### BACKGROUND OF THE INVENTION

In general, natural plants have been always seen as a source of health, freshness and wellbeing for people. For example, fresh flowers, are not only a lovely way of expressing sentiments and a pleasant way to decorate indoors places but also a source of healthier ingredients or medicine precursors (e.g. active ingredients). Their selection is based on season, colours and perfumes as well as for additional other ingredients (e.g. secondary metabolites). Fresh vegetables and fruits are attractive healthier food (important source of mineral and vitamins) for most people. These are mainly selected and consumed based on regional and seasonal availability, taste, appearance or nutrient content.

Unfortunately, fresh plants (e.g. vegetables, fruits as well as flowers) are perishable. Next to that, due to process of high quality selection, a lot of waste is constantly registered although the natural ingredients, like for instance active ingredients or secondary metabolites in the plant, are still usable. This plant waste can be smartly transformed into products before these are wasted, by using new technological approaches. To preserve their important ingredients, one needs to use invasive technologies which offer good preservation and stability of the valuable active components. These components can be further valorised as pharmaceutical, food supplements, nutraceuticals, cosmeceuticals or as cosmetic ingredients.

There are known technologies which aim to preserve natural ingredients and reported patents. U.S. Pat. No. 9,459,044B1 according to its abstract describes: “A freeze drying method includes decreasing pressure to a first vacuum pressure and, as a result of reaching the first pressure, a control system automatically activating a heater. The method includes sublimating solid water, increasing pressure to a second vacuum pressure greater than the first pressure, and, as a result of reaching the second pressure, the control system automatically deactivating the heater. As a result, a decrease in pressure, pressure-activated heater activation, material heating, water sublimation, and increase in pressure occur, accomplishing pressure-activated heater cycling. Another freeze drying method includes decreasing a temperature in a chamber to −50° F. or less using a refrigeration system with single-stage compression and sublimating solid water at a vacuum pressure. A freeze drying apparatus includes a chamber, a vacuum pump, a heater, and a control system programmed with instructions operable to accomplish pressure-activated heater cycling.”

U.S. Pat. No. 8,322,046B2 according to its abstract describes “A method of manufacturing heat-sensitive pharmaceutical powder is disclosed. The original pharmaceutical substances are dissolved in a solution or suspended in a suspension, which is sprayed through an atomizing nozzle and frozen in a cold gas phase or liquid nitrogen atomized directly in the spray-freeze chamber or gas jacket at the same time (for cooling purposes). The particles are freeze-dried at roughly atmospheric pressure in a down-stream fluid flow with exit filter thereby to remove moisture entrapped on or inside the frozen particles. The system has applicability for forming other powders.”

These known publications in general illustrate a process of encapsulating components like pigments, essential oils, proteins, antioxidants, etc. using air drying or freeze drying.

These known processes and products have been already commercialized based on these techniques. For instance, U.S. Pat. No. 8,637,104 according to its abstract describes “The present invention relates to a microencapsulate comprising microcapsules having a diameter of 0.1 μm to 25 μm, said microcapsules comprising: -a core particle having a diameter of 90 nm to 23 μm and containing at least 3% of the active component by weight of said core particle; and-a coating that fully envelops the core particle and containing at least 20 wt. % of a hydrophobic polymer selected from cellulosic ethers, cellulosic esters, zein, shellac, gluten, polylactide, hydrophobic starch derivatives, polyvinyl acetate polymers, polymers or copolymers derived from an acrylic acid ester and/or a methacrylic acid ester and combinations thereof; wherein the core particle contains a release trigger component and/or the coating contains a release trigger component, said release trigger component being selected from: -a water-swellable polymer having a water-uptake capacity at 37° C. and pH 7.0 of less than 20 wt. % and a water-uptake capacity at 37° C. and pH 2.0 of at least 50 wt. %; an-an edible salt having a water solubility at 37° C. and a pH of 7.0 of less than 1 mg/ml and a water solubility at 37° C. and a pH of 2.0 of at least 5 mg/ml; The microencapsulate of the present invention does not release the encapsulated active component when incorporated in water-containing foodstuffs, beverages, nutritional compositions or pharmaceutical compositions. Following ingestion, however, the active component is released rapidly.”

WO2011/087689 according to its abstract describes emulsion and double-emulsion based processes for preparing microparticles. US2004/156911, CN107788161, US2004/247624 and GB2388581 describe various other encapsulations.

Most of these technologies still use synthetic polymers as coating materials to protect the active ingredients. These are going to be banned in the near future due to their biodegradability issues as well as their toxicity in some cases.

### SUMMARY OF THE INVENTION

A disadvantage of prior art is that origin and authenticity and production quality are difficult to manage and to control.

Hence, it is an aspect of the invention to provide an alternative method for micro encapsulation, which preferably further at least partly obviates one or more of above-described drawbacks.

There is provided a method for microencapsulating a natural ingredient in an encapsulation material selected from a plant-based and inorganic encapsulation material, comprising:
mixing said natural ingredient for microencapsulation in a first liquid for providing a first mixture;
mixing said encapsulation material in a second liquid for providing a second mixture, said first liquid and second liquid being substantially immiscible, in particular displaying phase separation after being mixed;
preparing a microemulsion from said first mixture and said second mixture using mechanical shear forces;
spraying said microemulsion through a nozzle into a high-pressure vessel while drying it under continuously circulating supercritical CO2 to a powder having a particle size of between 0.1 and 100 microns.

In an embodiment, the particle size is between 0.1 and 10 microns.

In this sense, “particle size” means that the particles have a size distribution that has its mean, in particular its mean diameter of the particles, between 0.1 to 100 microns. Usually, reference is made to the smallest diameter of a particle.

In an embodiment, the particle size distribution has a width (standard deviation) (μ) which is less than 30% of the average particle size. In particular, the width is less than 20% of the average particle size. More in particular, the width is less than 10% of the average particle size. Specifically, the width is less than 50% of the average particle size.

The natural ingredients can for instance comprise active ingredients. These natural ingredients may also comprise secondary metabolites.

According to WIKIPEDIA:

“An active ingredient (AI) is the ingredient in a pharmaceutical drug or pesticide that is biologically active. The similar terms active pharmaceutical ingredient (API) and bulk active are also used in medicine, and the term active substance may be used for natural products. Some medication products may contain more than one active ingredient. The traditional word for the API is pharmacon or pharmakon (from Greek: φαρμακoν, adapted from pharmacos) which originally denoted a magical substance or drug.

The terms active constituent or active principle are often chosen when referring to the active substance of interest in a plant (such as salicylic acid in willow bark or arecoline in areca nuts), because the word ingredient in many minds connotes a sense of human agency (that is, something that a person combines with other substances), whereas the natural products present in plants were not added by any human agency but rather occurred naturally (“a plant doesn't have ingredients”). In contrast with the active ingredients, the inactive ingredients are usually called excipients in pharmaceutical contexts. The main excipient that serves as a medium for conveying the active ingredient is usually called the vehicle.”.

In an embodiment, the natural ingredients are derived, for instance extracted from, plant material. The natural ingredient can be said to be plant-based.

Wikipedia defines “plants” in the following way:

“Plants are mainly multicellular, predominantly photosynthetic eukaryotes of the kingdom Plantae. Historically, plants were treated as one of two kingdoms including all living things that were not animals, and all algae and fungi were treated as plants. However, all current definitions of Plantae exclude the fungi and some algae, as well as the prokaryotes (the archaea and bacteria). By one definition, plants form the class Viridiplantae (Latin name for “green plants”), a group that includes the flowering plants, conifers and other gymnosperms, ferns and their allies, hornworts, liverworts, mosses and the green algae, but excludes the red and brown algae.” In the current application, we include red and brown algae into the definition of “plants”.

In an embodiment, “natural ingredient” refers to compounds and compositions that are extracted from plant-based material. The natural ingredient may comprise a mixture of natural ingredients.

Microemulsions in particular are clear, thermodynamically stable, isotropic liquid mixtures of oil and water. In an embodiment, they comprise surfactant, frequently in combination with a cosurfactant. The aqueous phase may contain salt(s) and/or other ingredients, and the “oil” may actually be a complex mixture of different hydrocarbons and olefins. Often, microemulsions form upon simple mixing of the components and do not require the high shear conditions generally used in the formation of ordinary emulsions. Three basic types of microemulsions are direct (oil dispersed in water, o/w), reversed (water dispersed in oil, w/o) and bicontinuous.

In IUPAC definition, a microemulsion is a dispersion made of water, oil, and surfactant(s) that is an isotropic and thermodynamically stable system with dispersed domain diameter varying approximately from 1 to 100 nm, usually 10 to 50 nm.

In that definition, the average diameter of droplets in macro-emulsion (usually referred to as an “emulsion”) is close to one millimeter (i.e., 10-3 m). Therefore, since micro- means 10-6 and emulsion implies that droplets of the dispersed phase have diameters close to 10-3 m, the micro-emulsion denotes a system with the size range of the dispersed phase in the 10-6×10-3 m=10-9 m range.

In the current disclosure, microemulsion in an embodiment relates to drop sizes of about 0.1-10 micron.

Furthermore, the term “micro-emulsion” has come to take on special meaning. Entities of the dispersed phase are usually stabilized by surfactant and/or surfactant-cosurfactant (e.g., aliphatic alcohol) systems.

A process was developed to safely recover and preserve natural ingredients by means of scCO2 technology. Preservation of these natural ingredients is done via microencapsulation, or impregnation into, encapsulation material.

The encapsulation material can be made out of a natural material additionally offering an control-release property to the natural ingredient. It can be selected as such to confer hydrophilic or hydrophobic control-release and these can be originated in natural material. Examples of suitable basis for this natural material are selected from starch, starch-based, sugar, sugar-derived, wax, oil, fat, and mixture thereof.

Further natural materials are from solid natural material. In an embodiment, the solid based natural material is selected from natural clay, salt, mixtures of salts in the form of oxides and hydroxides, phytosillicates, phosphates like phytoxyapathites, and mixtures thereof.

The natural ingredient is mixed in a first liquid. The encapsulation material is mixed in a second liquid, and the first and second liquid are substantially immiscible. In particular, the first and second liquid display phase separation after being mixed. This means that after mixing, when put to rest, phase separation will occur. This phase separation will occur when the liquids are mixed without any emulsifier or other additional substance has been added. Usually, this means that one of the liquids is polar and the other one is nonpolar. It may also be regarded as one of the liquids being hydrophilic and the other one being hydrophobic. This phase separation is time dependent. When this occurs in a short time (short meaning not enough for further processing) like less than 2 hours, an emulsifier is needed.

The proposed solution is a two-stage process comprising a high pressure extraction stage followed by scCO2 encapsulation or impregnation stage. The solution could be applied to different natural ingredients, comprising secondary metabolites.

In an embodiment, natural ingredients are selected from pigment, colorant, antioxidant, essential oil, natural aroma, protein, enzyme, and a combination thereof.

Supercritical CO2 (scCO2) has been chosen as process for microencapsulation due to its non-toxicity, near-ambient critical temperature, high recovery and easy removal from the product once the process is completed.

Applicant developed a process based on CO2 techniques which can recover and subsequently preserve the valuable natural ingredients from plants. In an embodiment, these natural ingredients are selected from colorant, pigment, antioxidant, essential oil, wax, and combinations thereof. These natural ingredients can meet the constantly rising market demand of circular economy as well as the growing demand for natural ingredients from a natural source in contrast to synthetically obtained products. This is the result of constant consumer's demand, for instance with respect to health and wellbeing, as well as the legislation in force towards plastic and chemicals of oilfield petroleum origin in corroboration with circular recycling concept. Collecting and valorising valuable plant material can be done from different sources, for instance greenhouses, open fields, various markets, auctions, and combinations thereof. Sources of the natural ingredients are usually plant-based. These plant-based sources can in particular be selected from flowers, fruits and vegetables. The plant-based sources can be sorted based on natural ingredients and/or source (open field or green houses) and can be further treated to recover valuable natural ingredients. The natural ingredients are in particular selected from pigment, essential oil, anti-oxidant, protein, enzyme, and a combination thereof. These natural ingredients can be very sensitive towards air, oxygen, heat, moisture and light which prevents them of being used in a lot of products. Their stability issues can be corrected by protecting them against environmental degradation. The natural ingredient can be incorporated into the encapsulation material. In that sense, some natural ingredient may remain at the outer surface of agglomerates of encapsulation material. A protecting coating can enable a required products shelf life. Such a protecting coating using an encapsulation material can be as well selected from natural source, biodegradable source, or combinations thereof. It can be designed or selected to confer the natural ingredient a required stability and may ensure a long lasting preservation.

In order to coat a plant-based natural ingredient, first one needs to isolate the natural ingredient from the plant material. Literature mentions several ways to “extract” plant-based natural ingredient or natural ingredients (or any other natural ingredient, for that matter) from its natural source or sources. Most of these extraction methods are related to simple solid/liquid extraction combined with pressing and centrifugation in order to recover the active ingredients in a liquid concentrated form. In some of these processes, pressurized gases are also used as an extraction and transportation media (with or without the present of co-solvents).

Applicant developed a process which involves pressurized inert gasses. In an embodiment, these inert gasses are selected from CO2, N2 and a mixture thereof. This can be combined with solvent mixture to facilitate a better extraction of the natural ingredient from the plant cell. By utilizing pressurized inert gasses, for instance CO2, the plant cell turgor is equilibrated, thus the plant membrane becomes permeable, facilitating the dissolved natural ingredient or natural ingredients, in particular secondary metabolites, to diffuse out of the cell and into the solvent. The extract which is then rich in natural ingredient, in a particular embodiment a secondary metabolite, for instance selected from pigment, antioxidant, and a combination thereof, will be exposed to the inert gases, like a CO2 environment, which provides extra preservation and thus better stability to the extracted natural ingredient.

In an embodiment, the at least one natural ingredient originates from insect origin. The one or more insect-based natural ingredient can be combine with the earlier described plant-based material. In particular, enzymes may be retrieved as natural ingredient from insect base.

In an embodiment, the at least one natural ingredient originates from animal origin. The one or more animal-based natural ingredient can be combine with the earlier described plant-based material, the one or more animal-based natural ingredient can be combine with the earlier described insect-based material, or combinations thereof are possible. In particular, enzymes may be retrieved as natural ingredient from animal base.

To better preserve the natural ingredient or natural ingredients, it was found to be advantageous to microencapsulate the natural ingredient in order to restrict contact with external factors, for instance O2, high temperatures, light, humidity, and the like. These external factors can induce rapid degradation, thus lower activity and/or reduce shelf life. The microencapsulation process mainly comprises mixing encapsulation material of generally accepted natural origin which can be mixed with a stream, in particular a concentrated stream, of at least one plant-based natural ingredient. Subsequently, this stream is sprayed through a nozzle into a high pressured environment with an inert gas. This induces crystallization of encapsulation material for providing a coating around the at least one natural ingredient and sealing it or them, for instance providing increased preservation.

Selected plant material can be pre-dried or directly freshly used. The plant material can then be subjected to a following two stage treatment.

Stage One: Extraction

In this stage, one or more natural ingredients, for instance plant secondary metabolites, are recovered or extracted. The dry or fresh plant material is for instance placed in an autoclave where it is thoroughly contacted with a co-solvent or mixed with one or more solvents under high pressure. The solvents can be selected from alcohol, alcohol derivative, water, or a mixture thereof.

The ratio plant material to solvent is selected between 90%-10% w/w. Preferred is a ratio of 70%-20% w/w. Most preferred is a ratio of 60%-40% w/w.

The mixture is slowly brought to temperatures not exceeding 55-80° C. Preferably the mixture is brought to 40-55° C. Most preferred is a temperature of 45-50° C. Further, the mixture is pressurized with CO2 to a pressure which can equalize the turgor or a little bit exceed that. The pressure ranges are preferably between 80-350 bar. Preferably a pressure of between 80-180 bar is applied. Most preferably, a pressure of between 80 to 120 bar is applied.

The process will allow extraction of one or more natural ingredient, for instance one or more pigments, in a range of 0.05 to 0.8 mg/g. The extract can further contain one or more mono- and poly-saccharides which have also been co-extracted from the plant material. The extract is mixed with naturally occurring acids which can have an anti-oxidant or a preservative role for the extracted natural ingredient, like pigments. The anti-oxidants which are added can be in a solid form in ratios between 0.05% to 10%. These anti-oxidants in an embodiment are selected from one or more natural acid and a mixture thereof. In an embodiment, the anti-oxidant is selected from citric acid, tartaric acid, malic acid, and a mixture thereof. A further or alternative anti-oxidant is selected from Phenolic acid, flavonoid, carotenoid, vitamin E, ligand, and a mixture thereof.

A resulting solution is then dried (stripped of the solvent) using one or more pressurized inert gas. In particular, CO2 is applied. The appearance of the obtained product, usually a powder, is similar to dry pigment with a particle size of between 0.1 and 10 microns. The powder as it is obtained is hygroscopic when left longer at room conditions without being added into any formulation. The powder is recommended to be packed in sealed aluminium bags under inert gas blanket to prevent any degradation during storage.

The extract can be further mixed (in a liquid or solid form) with one or more encapsulation material. This encapsulation material can be selected for its function of further application. The encapsulation material, or mixture thereof, may convey extra stability or interaction properties which are further discussed in details in the discussion of a second stage.

Stage Two: Encapsulation & Impregnation

This stage focuses on preserving the received one or more natural ingredients, thus creating concomitant intermediate ingredients dedicated to the food or cosmetic industries.

Microencapsulation refers to a range of dosage forms which comprises encapsulation material, sometimes also referred to as matrix material, that can prevail and preserve natural ingredient by enclosing them into a relatively stable shell known as a capsule, or incorporating it in an amount of material. The process is conducted by means of pressurized emulsion or suspension CO2 technique. This technique is based on scCO2 spray drying process in which high pressure CO2 is brought in contact with the emulsion or suspension material which is pumped into the spraying vessel by utilizing a syringe-like system. The suspension or emulsion consists of one or more natural ingredients and the encapsulation material (also referred to as coating matrix) in a stabilized form. The pressurized fluid (supercritical fluid) will remove the solvent or will have an anti-solvent effect causing the precipitation of the encapsulation material around the natural ingredient. Function of crystallization properties and the interactions between the material in the encapsulation material causing the natural ingredient to be covered with different type of coatings or liposomes.

The main role of the encapsulation material is to shield the natural ingredient as well as to determine the release profile function of the application.

The encapsulation material can be composed of one or several components and is stabilized, if required, by means of surfactants or adjuvants to create a stable emulsion or suspension. The surfactants or adjuvants are added in a range of 0% to 10% w/w of the encapsulation material and can be non-ionic, cationic, anionic, amphoteric or amphiphile fatty materials originated in animal or plant tissues. These can be selected from lecithin, mono-glycerides, di-glycerides, fatty acids, mono or poly- glycerides with glycerol, and mixture thereof.

The encapsulation material is mixed in a minimum amount of first liquid, for instance water or solvent mixture, to create a sprayable emulsion. The required amount of first liquid depends on the solubility of the encapsulation material and the viscosity of the created emulsion or suspension which is to be easily pumpable.

The solution or suspension in an embodiment comprises a natural long and short chain poly-carbohydrates chosen from starch-like materials. Examples of these materials comprise wheat, maize (corn), peas, cassava, potatoes, beans, rice, tapioca, Other suitable encapsulation material are selected from long and short chain mono or poly saccharides. Examples of these are glucose, galactose, fructose, dextrose, ribose, trehalose, maltodextrin, maltol, etc. and mixture thereof. Other suitable encapsulation material comprises cellulose derivatives, for instance selected from methyl, ethyl cellulose type. Other encapsulation material comprises gums, for instance rosin, xanthan, guar, and mixtures thereof. Other encapsulation material comprises alginate, protein, and mixture thereof. These encapsulation materials can be dissolved in water. This frees the natural ingredient.

The concentration of the natural ingredient in a microcapsule can vary between 0.05% and 50%. In an embodiment the concentration of natural ingredient in the encapsulation material is between 1% and 45%. In a specific embodiment, the concentration of natural ingredient in the encapsulation material is 5% to 35%.

The appearance of the product is similar to any dry pigment. The shelf life is over 6 months.

The extract obtained in the first stage can also be used to be microencapsulated using one or more further materials used in the cosmetic industry.

Examples of these further materials are:

Oxides, for instance selected from rutile, zincite, periclase, hematite, maghemite, magnetite and mixtures thereof,

hydroxides for instance goethite,

carbonates for instance selected from calcite, magnesite and mixtures thereof,

sulphates for instance selected from gypsum, anhydrite and mixtures thereof,

chlorides for instance selected from halite, sylvite and mixtures thereof,

phosphates for instance hydroxyapatite,

and phyllosilicates, for instance selected from palygorskite, sepiolite, kaolinite, talc, montmorillonite, saponite, hectorite and mixtures thereof,

tectosilicates, for instance zeolites,

and mixture thereof.

These further material can be used to microencapsulate or serve as a matrix particle for incorporating one or more natural ingredients, for instance pigments, essential oils or anti-oxidants like polyphenols, which have been extracted from plant material following the procedure described above or obtained from other sources.

The process is conducted by means of pressurized gas technology utilizing inert gases like CO2 which are contacted with a suspension formed of the further material and one or more natural ingredient in ratios between 1% to 50% w/w. The obtained product has a dry powder appearance and is stable at room conditions. The water activity of the product is <0.4 mostly between 0.2 and 0.4.

Other processes are not so efficient in the sense of penetration of the natural ingredient in solid material. The high pressure gas drying has the advantage of putting in contact the solid crystalline frame with mixture formed of plant extract and pressurized gas. The pressurized gas has a very low surface tension and can easily penetrate through the solid matrix of the further material. As a consequence the water and/or solvent present in the solid matrix of the further material is completely removed simultaneously impregnating the further material with the natural ingredient like oil, pigment, anti-oxidant, enzyme, and mixtures thereof. This encapsulates the natural ingredient in the solid matrix of the encapsulation material. In this embodiment, small particles of the further material absorb the natural ingredient or surrounds the natural ingredients. The further material thus actually encapsulates amounts of natural ingredients, therefore labelling this as microencapsulation.

As discussed above, the invention relates to microencapsulation of natural ingredients originated in particular in an embodiment from plant material. Examples are pigment, essential oil, enzyme, antioxidants such as for instance phenols, polyphenol, and mixtures thereof, which are instable or can be subject to rapid degradation by direct contact with air, light or heat.

The invention further pertains to a microencapsulated natural ingredient, encapsulated in an encapsulation material derived from plant material.

In a particular embodiment, the microencapsulated natural ingredient is obtainable by the method described above. In a particular embodiment, said natural ingredient is selected from pigment, essential oil, antioxidant, enzyme and a combination thereof which is extracted from further plant material resulting in an first extract comprising said natural ingredient.

In a particular embodiment, the extraction comprises contacting said first extract with a supercritical fluid, in particular supercritical carbon dioxide. This extraction results in said natural ingredient. In a further embodiment, said resulting natural ingredient is emulsified in a liquid comprising said encapsulation material, resulting in an emulsion. In a more particular embodiment, said emulsion is sprayed into contact with a supercritical fluid, in particular supercritical carbon dioxide.

The invention further relates to a method for microencapsulating a natural ingredient in an encapsulation material, comprising:
providing a source material, in particular plant material;
contacting said source material with at least one solvent for extracting said natural ingredient from said source material;
contacting said solvent with said natural ingredient with a supercritical fluid, in particular supercritical carbon dioxide, for producing said natural ingredient;
mixing said natural ingredient for microencapsulation in a liquid comprising said encapsulation material, producing an emulsion;
spraying said emulsion through a nozzle into a high-pressure vessel while drying it under continuously circulating supercritical CO2 to a powder having a particle size of between 0.1 and 100 microns, in particular between 0.1 and 10 microns.

Some general example are presented below for some general applications.

### DOMESTIC APPLICATION EXAMPLE

Natural fragrances (natural ingredient) were microencapsulated using plant-based coating substances (encapsulation material) for use in fabric softeners. The encapsulates were able to disperse and remain stable in the fabric softener environment, and to hold the fragrance for several weeks after washing and release the it by mechanical force (i.e. friction on the textile or fabric). The encapsulation material was made from a combination of polysaccharides including lipophilic starch derived from corn, a cationic potato starch, and vegetable-based protein obtained from pea and soy beans. The ratio of different ingredients in the coating mixture was kept in a way to obtain a net positive surface charge in the final product. In this example, a starch/protein ratio of 65:35 was used. The starch mixture was dissolved in an aqueous basic solution at pH of 11. For a desire particle size of 50 μm, 0.2 Wt. % of a wheat-based bio-emulsifier was added to the coating mixture followed by the addition of fragrance. After thorough mixing, the protein was added to the mixture and stirred to make a thick paste. The emulsion was then micronized using a syringe pump at 150 bar, and spray-dried under supercritical CO2.

### COSMETICS EXAMPLE

Natural fragrances and essential oils obtained from plants (e.g. thyme), fruit (e.g. juniper berry) and flowers (e.g. lavender), as well as aroma extracted from cocoa butter and coconut oil (natural ingredients) were encapsulated at different fragrance loading from 5 to 35 Wt. % using the supercritical process indicated above. In this process, a combination of polysaccharides derived from rice, corn and potato were used as encapsulation material at various ratios. The choice of polysaccharide and their ratios depend on the application and is formulated so to achieve desired particle sizes ranging between 5 to 100 μm. In this example, an encapsulation material of mixture containing heat-resistant rice starch and corn-derived polysaccharide at 1:2 ratio was dissolved in distilled water, mixed with the fragrance oil extract followed by addition of a bio-emulsifier to ensure efficient dispersion of the oil. The bio-emulsifier used was derived from the wheat plant and added in ratios of 0.2 to 2 Wt. % depending on the fragrance loading in the final product and the desired size and roughness of the particles. With all ingredients combined, a microemulsion was made using a syringe pump connected to a nozzle at pressures between 50-150 bar. The emulsion was left to rest for at least one hour to test the stability and was then sprayed through a nozzle into a high-pressure vessel at varying flow rates where it dried under continuously circulating supercritical CO2.

There is further provided a method for microencapsulating a natural ingredient in an encapsulation material, comprising:

mixing said natural ingredient for microencapsulation in a first liquid for providing a first mixture;
mixing said encapsulation material in a second liquid for providing a second mixture, said first liquid and second liquid being substantially immiscible, in particular displaying phase separation after being mixed;
preparing an emulsion, in particular a microemulsion, from said first mixture and said second mixture;
spraying said emulsion through a nozzle into a high-pressure vessel while drying it under continuously circulating supercritical CO2 to a powder having a particle size of between 0.1 and 100 microns.

Using a microemulsion provides additional process efficiency. It may further produce finer powder, making the end product easier to use in preparations.

In an embodiment, the particle size is between 0.1 and 10 microns.

The terms “upstream” and “downstream” if used in this description relate to a process flow of material in a process. Thus, material is introduced into a process at a first position “upstream” in the process, and flows through the process relative to a second position within the process, which second position is then “downstream”.

The term “substantially” herein, such as in “substantially all emission” or in “substantially consists”, will be understood by the person skilled in the art. The term “substantially” may also include embodiments with “entirely”, “completely”, “all”, etc. Hence, in embodiments the adjective substantially may also be removed. Where applicable, the term “substantially” may also relate to 90% or higher, such as 95% or higher, especially 99% or higher, even more especially 99.5% or higher, including 100%. The term “comprise” includes also embodiments wherein the term “comprises” means “consists of”.

The term “functionally” will be understood by, and be clear to, a person skilled in the art. The term “substantially” as well as “functionally” may also include embodiments with “entirely”, “completely”, “all”, etc. Hence, in embodiments the adjective functionally may also be removed. When used, for instance in “functionally parallel”, a skilled person will understand that the adjective “functionally” includes the term substantially as explained above. Functionally in particular is to be understood to include a configuration of features that allows these features to function as if the adjective “functionally” was not present. The term “functionally” is intended to cover variations in the feature to which it refers, and which variations are such that in the functional use of the feature, possibly in combination with other features it relates to in the invention, that combination of features is able to operate or function. The word “functionally” as for instance used in “functionally parallel” is used to cover exactly parallel, but also the embodiments that are covered by the word “substantially” explained above. For instance, “functionally parallel” relates to embodiments that in operation function as if the parts are for instance parallel. This covers embodiments for which it is clear to a skilled person that it operates within its intended field of use as if it were parallel.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

As will be clear to the person skilled in the art, the invention is not limited to methods of operation or devices in operation.

It should be noted that the above-mentioned embodiments illustrate rather than limit the invention, and that those skilled in the art will be able to design many alternative embodiments without departing from the scope of the appended claims. Use of the verb “to comprise” and its conjugations does not exclude the presence of elements or steps other than those stated in a claim. The article “a” or “an” preceding an element does not exclude the presence of a plurality of such elements. The mere fact that certain measures are recited in mutually different dependent claims does not indicate that a combination of these measures cannot be used to advantage.

The invention further applies to an apparatus or device comprising one or more of the characterising features described in the description and/or shown in the attached drawings. The invention further pertains to a method or process comprising one or more of the characterising features described in the description and/or shown in the attached drawings.

The various aspects discussed in this patent can be combined in order to provide additional advantages. Furthermore, some of the features can form the basis for one or more divisional applications.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIG. 1 shows extracted natural ingredient, here pigment, in the first liquid;

FIG. 2 shows the pigment dried in an scCO2 process;

FIG. 3 shows emulsifying of pigment in the encapsulation material in the second liquid;

FIG. 4 shows the dried, microencapsulated natural ingredient;

FIG. 5 shows the emulsion of the natural ingredient and encapsulation material of example 5;

FIG. 6 shows the microencapsulated natural ingredient of example 5;

FIG. 7 shows the emulsion of natural ingredient and encapsulation material, here lutein oil and rice starch PO4;

FIG. 8 shows a SEM picture of the emulsion;

FIG. 9 shows a SEM picture of the microencapsulated natural ingredient;

FIG. 10 shows a picture of the microencapsulated natural ingredient

FIG. 11 shows the encapsulated natural ingredient of example 3.

### DESCRIPTION OF PREFERRED EMBODIMENTS

In the examples below, extraction using supercritical CO2, carbon dioxide, was used, in some examples at different stages. In these examples, a natural ingredient was microencapsulated in an encapsulation material of natural origin, in particular plant-based or retrieved or extracted from plant material.

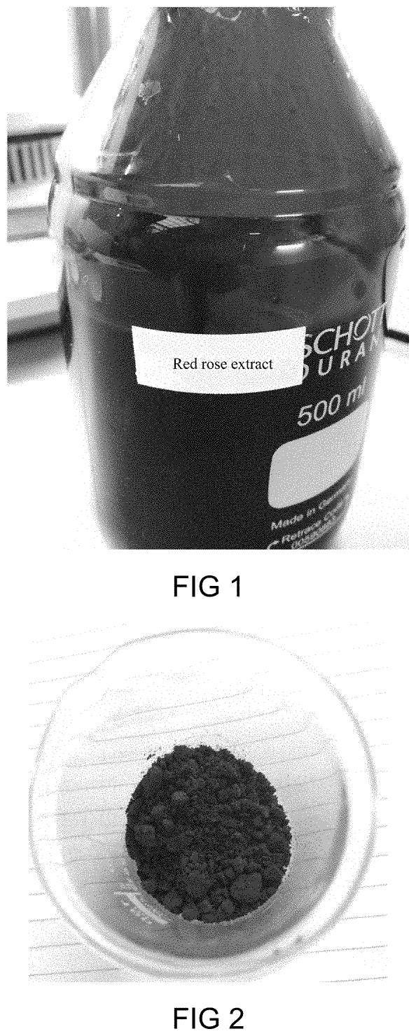

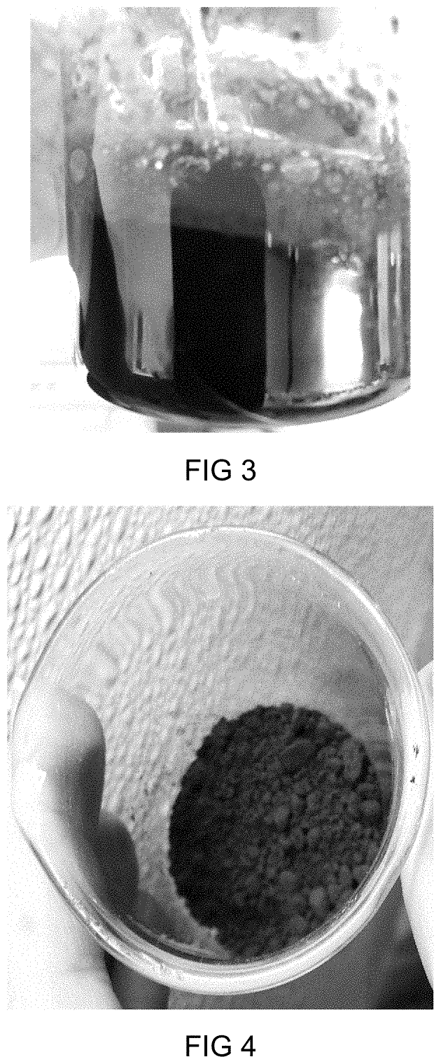

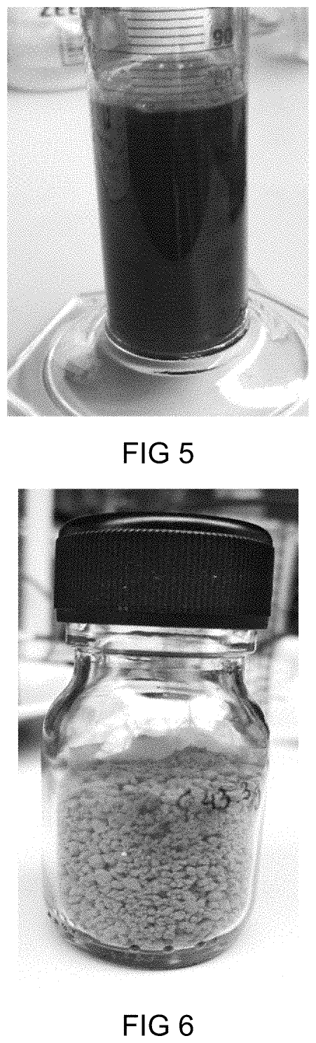

FIGS. 1-4 show intermediate results and end result of a microencapsulation method in which a natural ingredient is encapsulated in an encapsulation material. In FIG. 1, the result of extraction of a natural ingredient, here rose pigment, in s solvent is shown. In FIG. 2, the result of solvent removal through exposure to supercritical CO2 is shown. In FIG. 3, the natural ingredient, the pigment in this example, is emulsified with an encapsulation material. In FIG. 4, the result of processing the emulsion using supercritical CO2 like for instance illustrated in example when injected into an autoclave inn which supercritical CO2 is circulated,

### EXAMPLE 1: EXTRACTION OF A NATURAL INGREDIENT

We used supercritical CO2 (scCO2) to improve extract efficiency of plant pigments.

From fresh cut roses, here red roses cultivated in green houses, flower heads or capitulum were washed to remove possible pesticides and the petals (ray floret) were removed from the rest of the flower and dried at room conditions for 1-2 weeks.

The dry petals (aw<0.4) were subjected to milling (using here a coffee milling machine) to obtain a dry powder which was mixed and soaked into a solution made of ethanol/water in a ratio 30/70% w/w. The amount of dry petal versus solvent mixture was kept at 0.04 g/mL. The mixture was placed into a double wall heated autoclave equipped with a stirrer. The process was kept between 1 to 2h at constant conditions of 49° C. and 100 bar.

The process was stopped after 1h and the soaked rose petals were further centrifuged for 15 min at 4500 rpm and at 10° C. to maximize the solvent recovery. The solution is further sieved using a 90 micron mesh and then concentrated by vacuum evaporation in a rotary evaporator at Pmax 100 mbar, P min 20 mbar, T=40-45° C. with a condensed water temperature at 2-25° C. and a rotation speed of 7. The solution is concentrated approx. 14 times. This concentration step is mainly necessary for economic reasons.

The concentrated solution is subsequently spray dried by scCO2 spray into a closed autoclave. The process as such was further described by the U.S. Pat. No. 8,637,104B2 as follows. A high pressure vessel was 18 litres in volume. Before pumping the solution into the vessel, the vessel was pressurized with carbon dioxide to 120 bar using a membrane pump (Orlita®). The carbon dioxide entering the high pressure vessel was heated to 40° C. through an oil-heated jacket. The vessel was equipped with a stainless steel sintered filter, which was mounted at the bottom of the vessel. Over the sintered filter a single use paper filter material was used to capture all formed powder material. Dispersion was added to the vessel at a rate of 0.1-20 ml/min, most used 0.5-10, preferred 1-5 mL/min. Carbon dioxide was simultaneously added to the top of the vessel through the outer passage of the coaxial nozzle at a recirculation rate of 500 kg/hour. The carbon dioxide was removed from the autoclave via a tube situated at the bottom of the vessel and send to the recovery system where was passed over a zeolite bed. This ensure a continuous recirculation of the CO2 during the spray drying process. The pressure in the vessel was controlled via a valve connected to that tube. After completion of the spraying was completed, the vessel was further flushed with fresh CO2 for another 20 min before pressure was released. A powder was collected from the filter at room conditions.

The collected dry pigments can be stored for later use or further re-dispersed into a mixture of encapsulation material as matrix materials used for coating or impregnation.

### EXAMPLE 2: ENCAPSULATION OF NATURAL LUTEIN EXTRACT

Core particle loading: Lutein oil extract (olive oil based extract) 3.2 g oil with 60% lutein extract in the oil. The extract was obtained from dried marigold flower petals using scCO2 and olive oil as co-solvent in a process according to example 1.

Coating material: 2.5 g Rice Starch PO4 (Agrana) suspended in 10 g distilled water in the following way. Warm the solution to 60° C. for 1 h, add 5% (0.25 g) MD-5 potato starch (Avebe) and 95% (4.75 g) MD-20 potato starch (Avebe) and bring the solution to 40° C.

Subsequently, 3.2 g lutein oil is added into the coating material solution under vigorous stirring using an Ultra-Turrax (IKA® Ultra-Turrax).

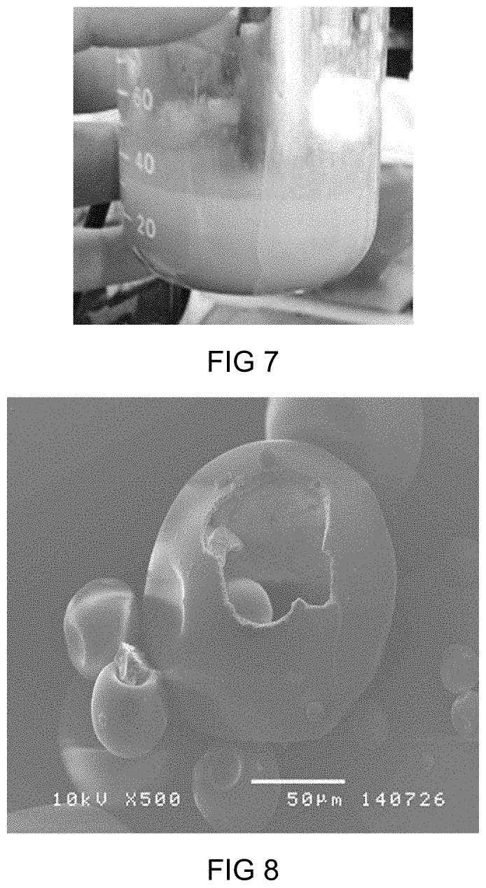

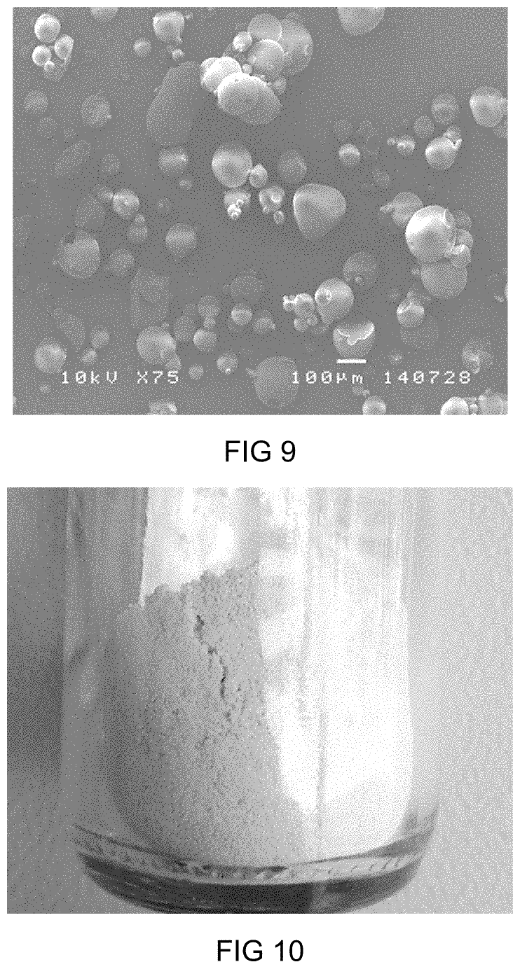

The homogenized emulsion, shown in FIG. 7, was sprayed dried through a 1650/64 nozzle (Spraying Systems) by means of the scCO2 process similar to example 1. To that end, the emulsion was injected in a scCO2 closed autoclave provided with a scCO2 recirculation similar to the process describe in example 1. The resulting microencapsulated natural ingredient is shown in FIG. 10, showing a dry, powdery product. In FIGS. 8 and 9, SEM picture are shown of the microencapsulated natural ingredient. Most particles are smaller than 100 micron.

### EXAMPLE 3. ENCAPSULATION AND STABILIZATION OF NATURAL ANTHOCYANIN EXTRACT

A preparation is made of a solution of 4 g carrageenan in one part (16 mL) castor oil. The solution is slowly heated up under continuous stirring until we obtained an homogenized solution. Leave it to cool down to 35° C. and add 30% wt (on total dry weight) anthocyanin dry powder pigment and stir vigorously using an Ultra-Turrax until the solution is homogenized.

Separately make a 5 parts distilled water (80 mL) in which 2 g Rice starch PO4 is added. The solution is slowly heated under stirring until a homogenised mixture is obtained.

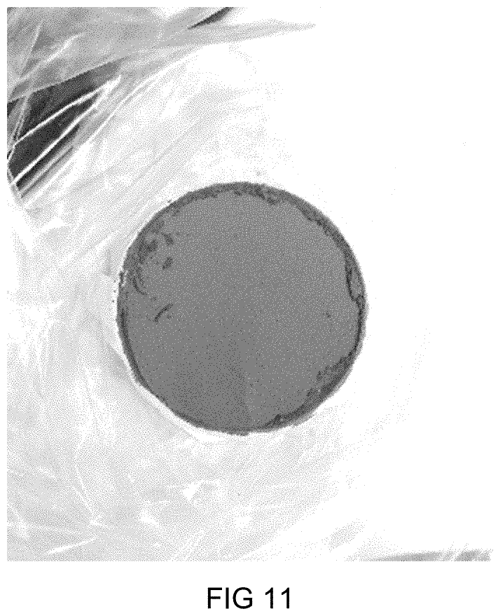

Pour the water phase over the oil phase and mix the emulsion vigorously with an Ultra-Turrax for 5 min. The homogenize emulsion was further spray dried using a scCO2 process similar to Example 1. The result is a dry, powdery encapsulated carrageenan, shown in FIG. 11.

### EXAMPLE 4. ENZYMES FOR COSMETIC AND LAUNDRY APPLICATIONS

The encapsulation of enzymes is done to extend the enzyme activity and controlled-release of enzyme upon scrubbing force when used in laundry liquids as well as other cleaning products.

In this example, β-galactosidase as one of the most important enzymes in food and pharmaceutical products, was immobilized by spray-drying with supercritical CO2 using a combination of plant-based biopolymers and polysaccharides including modified chitosan, gums such as Arabic gum, modified cationic starch derived from corn, lipophilic starch derived from rice and high-purity alginates. The coating mixture was formulated to yield a certain particle size ranging from 30 to 50 μm and was tested for the stability in the acidic environment of vanish gel and washing detergents (pH 3 to 4).

The spray-drying emulsion contained the enzyme at 0.5-2 Wt. % concentration, BSA (bovine serum albumin) to stabilize the enzyme, and the coating mixture. The emulsion was spray-dried into a high-pressure supercritical CO2 vessel with CO2 being continuously recirculated through the system similar to example 1. The obtained microencapsulates were characterized for the particle size and morphology (using Scanning Electron Microscopy, SEM) and the enzyme activity (using UV-VIS spectrophotometry).

### EXAMPLE 5 ENCAPSULATION AND STABILIZATION OF NATURAL ANTHOCYANIN EXTRACT

Preparation of solution 1: Disperse 22.4 g of Tapioca starch (fine powdered starch, commercially available) in 448 mL of distilled water. Place the suspension under slow stirring for 1 h. Maintain continuous stirring until the starch is completely gelatinized. The solution temperature should reach around 90° C. Let the starch solution to cool down to room temperature while making the second solution.

Preparation of solution 2: Use 100 g Tartaric acid (Sigma) in 100 g ethanol to stir well at 60° C. until the acid is completely dissolved. Bring the solution temperature to 40° C. and add 10 g of anthocyanin extract. Use an ultrasound device for 15 min for achieving a well-dispersed dispersion and dissolving of the pigment into the tartaric solution.

Slowly pour (dropwise) solution 2 over solution 1 under vigorous stirring. The stirring is ensured by an Ultra-Turrax. Add NaK-tartrate (1 g in 50 mL water) solution and dilute the entire mixture with 10 mL fresh ethanol. The solution/suspension was left for de-aeration at 20° C. overnight.

Before using it, the homogenized suspension was spray dried using a scCO2 process. The conditions used were similar as described in example 1.

### OTHER EXAMPLES

Anthocyanin Sepiolite Citric Acid

21 g Sepiolite was mixed with 10.1 g dry anthocyanin produced from Naomi red rose extract. Then 1.5 g of citric acid was added. The solid powder was mixed with 127 g solution made out of 80mLH2O and 47 mL solution (30gH2O and 27gC2H5OH). The red solution was spray dried by means of scCO2 using 120 bar and 40 C with a solution flow rate of 3 mL/min and a CO2 flow rate of 40 kg/h.

Enclosed the powder obtained with a particle size between 3-5 microns.

MD20, MD5 Rice starch

The dry anthocyanins from Naomi red rose encapsulated in a mixt capsule obtained form MD20,MD5 and rice starch. The process was made utilizing the following recipe:

8 g anthocyanin dry powder (previously obtained via sc02 draying of extarted obtained from Naomi Red Roses) was mixt with 8.4 g MD20, 2.1 g MD5 and 5.2 g rice starch and 0.02 Bioemulsifier. 50 mL distilled water was added over the mixture. Pigmet represents 24.6 wt % of the mixture (solid ratios). The Coating/pigment ration was 2.

The obtained aqueous solution was spray dried utilising a scCO2 spray drier under the following conditions: 120 bar, 40C, 1 mL/min solution flow, 800/1000 mbar CO2 flow.

The dry encapsulated anthocyain product was collected from the filter and placed into a glass bottle. See photo above).

The encapsulation yield was between 95-98% assest by measuring the free anthocyain existing outside the capsule Named PL) and the total anthocyanin present in the product. The difference deterin the encapsulated material. The values for PL were between 0.09 and 0.03 which indicates very small amount of free anthocyanin present in the product. The rest was encapsulated. For determining the PL values a sample of dry powder containing encapsulated anthocyains was disperse into ethanol. The resulted solid was centrifugated and filtered. The liquid was spectrofotometrically measured. For determining PT a similar amount of powder was totally dissolved into an aqueous solution of pH=2. The total anthocyain was determined spectrophotometrically. The difference was indicating the anthocyanin amount which was practically encapsulated.

Using similar coating material but different biochromes, dry red radish extract, the encapsulation anthocyanin has a different red shade colour.

It will also be clear that the above description, examples and drawings are included to illustrate some embodiments of the invention, and not to limit the scope of protection. Starting from this disclosure, many more embodiments will be evident to a skilled person. These embodiments are within the scope of protection and the essence of this invention and are obvious combinations of prior art techniques and the disclosure of this patent.

1. A method for microencapsulating a natural ingredient in an encapsulation material selected from a plant-based and inorganic encapsulation material, comprising:
mixing said natural ingredient for microencapsulation in a first liquid for providing a first mixture;
mixing said encapsulation material in a second liquid for providing a second mixture, said first liquid and second liquid being substantially immiscible;
preparing a microemulsion from said first mixture and said second mixture using mechanical shear forces;
spraying said microemulsion through a nozzle into a high-pressure vessel while drying it under continuously circulating supercritical CO2 to a powder having a particle size of between 0.1 and 100 microns.

2. The method of claim 1, wherein said first liquid is polar and said second liquid is nonpolar.

3. The method of claim 1, wherein said first liquid is nonpolar and said second liquid is polar.

4. The method of claim 1, wherein said first liquid is hydrophilic.

5. The method of claim 1, wherein said first liquid is hydrophobic.

6. The method of claim 1, wherein said encapsulation material is selected from encapsulation material which crystallizes when sprayed in said high-pressure vessel with supercritical CO2, in particular at a pressure of 80-350 bar.

7. The method of claim 1, wherein said natural ingredient is extracted from plant-base material.

8. The method of claim 7, wherein said natural ingredients are retrieved from plant material and are selected from pigment, essential oil, antioxidant, enzyme and a combination thereof.

9. The method of claim 1, wherein said encapsulation material is substantially derived from plant-based material.

10. The method of claim 1, wherein said encapsulation material is substantially derived from plant-based material, or said encapsulation material is selected from natural or mineral clay, salt, mixtures of salts in the form of oxides and hydroxides, and mixtures thereof, and of mixtures of any of these encapsulations material.

11. The method of claim 1, wherein said encapsulation material is substantially derived from plant-based material, wherein said encapsulation material is selected from:
natural long and short chain poly-carbohydrates,
long and short chain mono or poly saccharides type,
cellulose derivatives;
gums.

12. The method of claim 1, wherein said encapsulation material is selected from:
oxides;
hydroxides;
carbonates;
sulfates;
chlorides;
phosphates;
phyllosilicates;
tectosilicates.

13. The method of claim 1, wherein said encapsulation material is substantially derived from plant-based material, wherein said encapsulation material is selected from polysaccharides including lipophilic starch derived from corn, from cationic potato starch, vegetable-based protein obtained from pea and soy beans from rice, from corn and from potato, from heat-resistant rice starch from corn-derived polysaccharide, and a mixture thereof.

14. The method of claim 1, wherein a source material for said at least one natural ingredient is contacted with at least one solvent for extracting said at least one natural ingredient from said source material, and said solvent with said at least one natural ingredient is contacted with a supercritical fluid for producing said at least one natural ingredient.

15. A powder having a particle size of between 0.1 and 100 microns, of a microencapsulated natural ingredient, encapsulated in an encapsulation material derived from plant material, obtainable by the method according to claim 1, resulting in said microencapsulated natural ingredient.
